(19) United States
(12) Patent Application Publication
(10) Pub. No.: US 2021/0393796 A1
(43) Pub. Date: Dec. 23, 2021

(54) Methods for sustainable human cognitive enhancement

(71) Applicant: Cognigenics, Inc., Petaluma, CA (US)

(72) Inventor: John Lawrence Mee, Stuart, FL (US)

(73) Assignee: Cognigenics, Inc., Petaluma, CA (US)

(21) Appl. No.: 17/173,113

(22) Filed: Feb. 10, 2021

Related U.S. Application Data
(63) Continuation-in-part of application No. 15/970,037, filed on May 3, 2018, now abandoned.

(51) Int. Cl. A61K 47/69; A61K 31/713; A61K 38/46

(52) U.S. Cl. CPC A61K 47/6901; A61K 38/465; A61K 31/713

(57) ABSTRACT

A method of achieving sustainable, general-purpose cognitive enhancement comprising administering a gene-editing endonuclease complexed with a neuron-targeting vector and a synthetic guide RNA to lower the population of 5-hydroxytryptamine 2A receptors in the brain.

### CROSS REFERENCE TO RELATED APPLICATIONS

This application is a continuation in part of and claims priority to U.S. patent application Ser. No. 15/970,037, filed May 3, 2018, and entitled “Method for sustainable human cognitive enhancement”, the entire disclosure of which is incorporated herein by this reference.

### FIELD OF THE INVENTION

The present invention relates generally to human genetic engineering, and more particularly to the application of genetic engineering methods and techniques to expand human cognitive capacity.

### BACKGROUND OF THE INVENTION

The present invention generally relates to a method for improving human cognitive performance through the application of genetic engineering.

Although the initial human applications for genetic engineering are in medicine, the technology's greatest potential lies in human enhancement. Specifically, genetic engineering can be applied to improve human abilities in four ways:

1. Cognitive: Cognitive faculties such as awareness, concentration and/or mental acuity.
2. Anatomy: Physical attributes such as strength, agility, beauty, grace and/or stamina.
3. Longevity: Elements of long life including wellness, immunity and/or metabolism.
4. Talent: Mental, physical and/or emotional talents.

The current invention pertains to the first category, cognitive enhancement. The applications of genetic engineering methods to raise cognitive abilities can improve the lives of large numbers of people and expand their capacity to make meaningful contributions to society.

### SUMMARY OF THE INVENTION

In one embodiment, the invention provides a genetic cognitive enhancer which delivers any one or more of persistent higher states of awareness, concentration, focus, clarity, mental acuity, mindfulness and/or creativity.

In another embodiment, the invention provides a safe and effective genetic cognitive enhancer which delivers the aforementioned results from a single, one-time application. In other embodiments, multiple applications may be used to provide any one or more of the aforementioned results.

In another embodiment, the invention provides a genetic cognitive enhancer which does not affect the germline.

In a more specific embodiment, the invention provides methods of achieving sustainable, general-purpose cognitive enhancement comprising administering a gene-editing endonuclease complexed with a neuron-targeting vector and a synthetic guide RNA to lower the population of 5-hydroxytryptamine 2A receptors in the brain of a subject such as a human subject.

As those in the pertinent art understand, reducing a neuron's 5-hydroxytryptamine 2A receptor population raises its electrical resistance, thereby lowering its electrical conductivity and excitability. Higher electrical resistance in neurons decreases brain current density and attenuates brainwave activity. Diminished brainwave activity has been scientifically correlated with higher states of awareness, concentration, focus, creativity and mental acuity.

One aspect of the present invention provides a catalytically-active gene editing endonuclease which deletes gene HTR2A, which controls the expression 5-hydroxytryptamine 2A receptors in neurons.

Another aspect provides a catalytically-inactive gene editing endonuclease which blocks the RNA transcription of gene HTR2A.

A further aspect provides a vector which transfects neurons.

Yet another aspect provides a synthetic guide RNA which navigates the gene editing endonuclease to gene HTR2A on chromosome 13.

Another aspect provides a method of calculating dosages for genetic cognitive enhancements.

A further aspect provides a psychological screening method for determining suitable candidates for genetic cognitive enhancement.

### DESCRIPTION OF THE DRAWINGS

FIG. 1 is an illustration of a neurowave flowing through a series of neurons;

FIG. 2 is an illustration comparing neurons in series with transistors in series;

FIG. 3 is a graph depicting a typical neuron's pulse rise time;

FIG. 4 is an illustration of neurowave voltage and frequency;

FIG. 5 is a diagram explaining neuron electrodynamics using a resistor-capacitor network example;

FIG. 6 is a detailed illustration of the electrical characteristic of a neurowave;

FIG. 7A is the first page of a flowchart diagram illustrating a method for achieving sustainable human cognitive enhancement using genetic engineering;

FIG. 7B is the second page of a flowchart diagram illustrating a method for achieving sustainable human cognitive enhancement using genetic engineering;

FIG. 8 is an illustration of an editing strategy for deleting the HTR2A gene; and

FIG. 9 is an illustration of a knock-in strategy for stopping transcription of the HTR2A gene.

### DETAILED DESCRIPTION

### I. Definitions

Neurowaves: Brainwaves are composed of millions of tiny, cellular-level electromagnetic waves which travel through neurons. This application refers to these neuron-level electromagnetic waves as “neurowaves.”

### II. Overview

### 1. Brain Currents

As those skilled in the art understand, a moving electrical current generates an electromagnetic wave (per Ampere's Law). Flowing electrons in the brain generate brainwaves. When the flowing electrons slow down, so does brainwave activity.

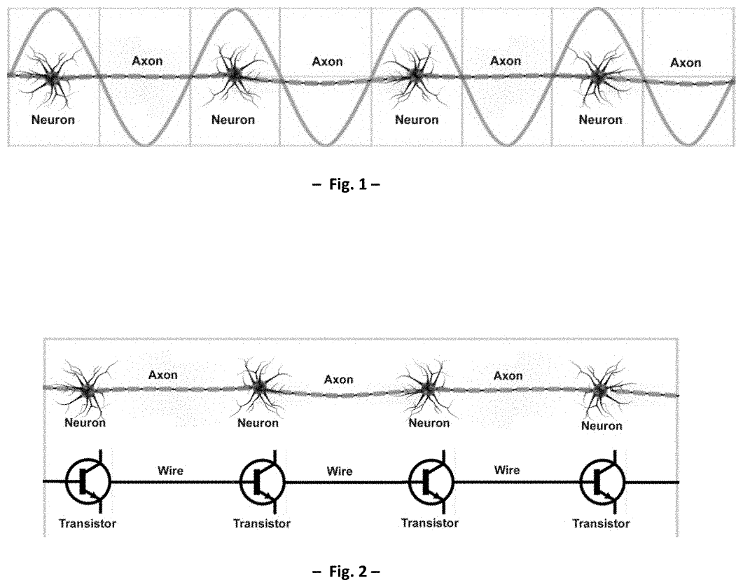

FIG. 1 shows a neurowave traveling through a series of neurons. Every neurowave has a corresponding flow of electrical current which runs through neurons in the brain.

Brain currents flow through neurons at different rates, depending on the neuron's physical properties. Neurons which have higher electrical resistance impede the flow of current, while neurons with lower resistance conduct current more readily.

When the flow of a brain current is impeded, its associated brainwave slows down. Slower brainwaves exhibit lower overall activity per second.

### 2. Neuron Electrodynamics

As shown in FIG. 2, the axon filaments connecting neurons resemble wires connecting transistors in a series. From a functional perspective, a neuron is a switching mechanism for electrical impulses, much like a transistor. Computers are made of transistors connected by wires, while brains are made of neurons connected by axons. In a computer, transistors act as logical switches which send electrical pulses along conducting wires. In the brain, neurons act as logical switches which send electrical pulses along interconnecting axons.

Research at Yale and Stanford has shown that flowing electrons in the brain's neural networks are accompanied by tiny electromagnetic waves typically measuring 55 millivolts and 5 nanoamperes. This relatively large voltage compared to the small amount of current is necessary to overcome the resistance of the brain's electro-chemical circuits, which is very high compared to ideal conductors like copper or gold.

Brainwave frequencies, conventionally expressed as a number between 1 to 40 Hertz, measure the average number of neuron conversations per second. When it takes longer for one neuron to talk to the next one, there are fewer neuron conversations in any given unit of time, and brainwave activity diminishes.

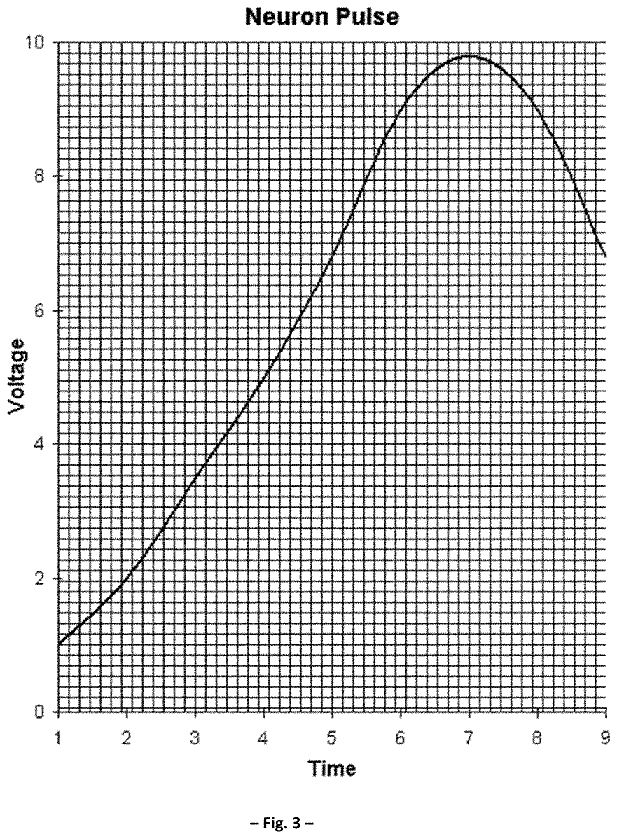

As those skilled in the art understand, neurons and transistors alike transmit information as pulses of electromagnetic potential, or “voltage.” Before a neuron can send a pulse, it first must build up the energy for the pulse. FIG. 3 illustrates the time a neuron takes to accumulate this voltage, which is called pulse rise time.

Once the energy in the neuron reaches the “threshold value” necessary to send a pulse (i.e., the top of the curve shown in FIG. 3), a spurt of energy is released from the neuron. This pulse is often called a neuron “spike,” and its voltage is what brainwave measuring devices sense and convert into brainwave frequencies. For example, an average rate of 30 “spikes” per second would be reported by EEG as a brainwave frequency of 30 Hertz.

The “spike” of flowing electrons is transmitted from one neuron to the next one across the synaptic gap via neurotransmitter receptors. The 5-hydroxytryptamine 2A receptors are one such type of receptor.

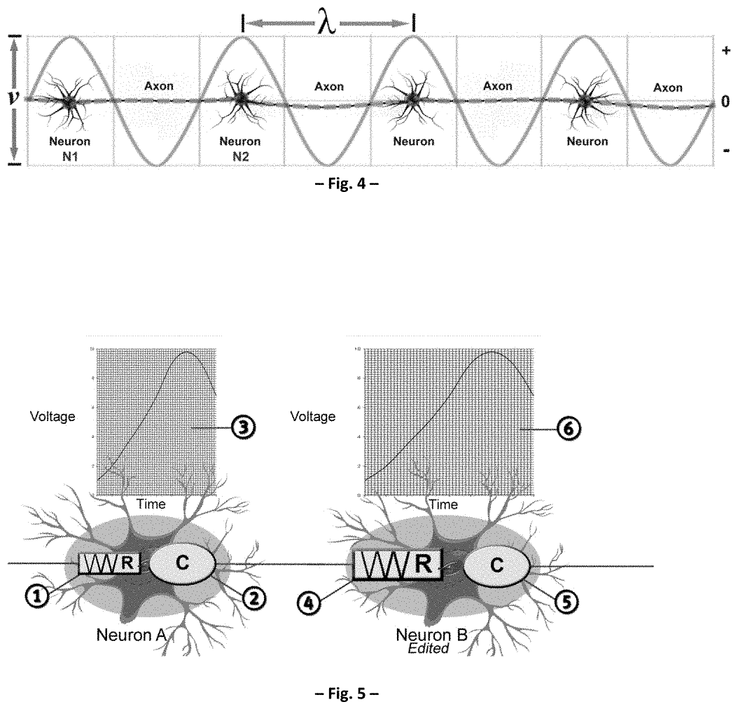

FIG. 4 shows four neurons connected in a series by axons. Each neuron emits a pulse, which collectively form an electromagnetic wave or “neurowave.” The neurowave is shown plotted against voltage grid v.

As illustrated in FIG. 4, the neurowave's wavelength X is equal to the time between peaks in the wave. This can be expressed mathematically as λ=P+A, where:

λ=Wavelength of neurowave;
P=Neuron pulse rise time; and
A=Axon transmission time.

In FIG. 4, the wave is energized when Neuron N1 fires, then decays over the axon transmission until it is re-energized when the next Neuron N2 fires.

To further clarify how neurons generate electromagnetic waves, consider the neuron's electrical counterpart inside a computer: the “RC circuit” (resistor/capacitor). In electrical engineering, networks of resistors and capacitors are utilized to convey signals comprised of electromagnetic waves. A capacitor stores electrons which enter it like a reservoir holds water behind a dam. When the accumulated charge in a capacitor reaches its “threshold value,” it discharges, and all the stored electrons in the capacitor flow over the dam, creating an electromagnetic pulse. In an RC circuit, flowing electrons will enter a capacitor at a rate determined by the size of a resistor placed in front of the capacitor. A larger resistor will slow the electrons down; lengthening the amount of time it takes the capacitor to fill up.

As those skilled in the art understand, in the brain, networks of neurons, acting as both resistors and capacitors, convey signals composed of neurowaves. As a resistor, the neuron funnels incoming ions from the axon through a limited number of receiving channels called receptors. The number of open input channels a neuron has to receive incoming electrons determines its resistance. The more open channels, the less resistance, and the faster it fills. Fewer open channels slow down the flow of incoming ions much like a bottleneck impedes the flow of traffic on a freeway. The fewer open channels, the more resistance, and the slower it fills.

As a capacitor, the neuron stores and holds the ions, like a reservoir holds the water behind a dam. When the capacitor accumulates enough charge to exceed its threshold value, all the stored electrons in the reservoir flow overflow the dam. As shown in FIG. 5, neuron B, which is edited to have a higher resistance, takes more time to fill up and generate a pulse than unedited neuron A.

Referring to FIG. 5, unedited neuron A has a resistance 1 which regulates the flow of ions into the neuron's capacitance reservoir 2, yielding a voltage accumulation rise shown time in graph 3. Edited neuron B has a larger resistance 4 regulating the flow of ions into the neuron's capacitance reservoir 5, yielding a slower build up of voltage as shown in graph 6.

Neurons can be edited to raise their resistance by reducing the number of input channels, or “receptors” they have. Neurowaves are comprised of thousands of individual neuron pulses which are emitted by the neurons over which the wave travels. Slowing down even one of these pulses will change the frequency signature of the wave.

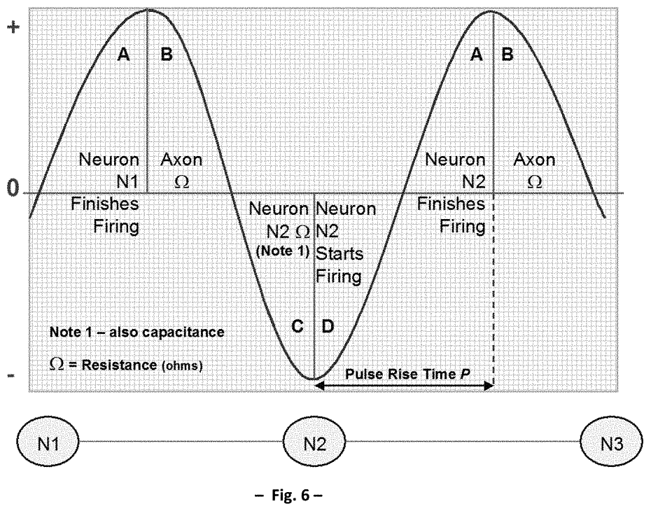

Specifically, the electrical characteristics of the neurowave can be divided into four quadrants: A, B, C, and D, as shown in FIG. 6.

Quadrant A: Neuron N1 releases its pulse signal at the peak of quadrant A. The high voltage at the peak of the wave impels the signal across the axon.
Quadrant B: The signal's voltage diminishes in quadrant B above as it travels across the resistance of the axon.
Quadrant C: Negatively-charged electrons meet Neuron N2's resistance, and gather in the capacitance reservoir of Neuron N2.
Quadrant D: Neuron N2 begins to fire, causing the process to repeat itself.

### 3. Conclusions

Raising neuron resistance decreases brain current density and brainwave activity, as recapped below:

### a) Brain Current

Brain current can be expressed by Ohm's Law

$$I = \frac{E}{R}$$

where:
I=Brain current
E=Brain voltage
R=Resistance of neuron

Hence, raising resistance R decreases brain current I.

### b) Brainwave Activity

In a neurowave wavelength expressed λ=P+A, where:
λ=Wavelength of neurowave;
P=Neuron pulse rise time; and
A=Axon transmission time: Assuming fixed axon length, wavelength is a direct function of pulse rise time. Pulse rise time lengthens as neuron resistance rises. Hence, raising neuron resistance increases a neurowave's wavelength, decreasing the number of neuron spikes per unit of time (which collectively comprise brainwave activity).

### 3. Brainwaves and Consciousness

In developing genetic cognitive enhancement technology for raising conscious awareness, mental acuity, focus, attention and/or cognitive performance, a precise understanding of the relationship between brainwaves and consciousness is required. Typically, brainwaves are divided into 4 categories:

1. Beta State: (16 to 30 Hz) Beta waves are associated with the alert mind state of the prefrontal cortex. This is a state of the working or thinking mind: analytical, planning, assessing and categorizing. Excess beta wave activity is associated with stress.
2. Alpha State: (9 to 15 Hz) Alpha waves are associated with relaxation, creativity, imagination, lucidity, reflection, and peacefulness.
3. Theta State: (4 to 8 Hz) Theta waves are typical of deeper states of consciousness, such as meditation, and are correlated with stronger intuition and greater capacity for clarity, visualization and problem-solving.
4. Delta State: (1 to 3 Hz) Expert meditators, such as Tibetan monks, can reach delta waves in an alert, wakened state, but most people experience them during deep sleep.

Hundreds of scientific experiments show that the lower frequency alpha and theta brainwave states are correlated with reduced stress and improved mental acuity, concentration and cognitive performance.

Twenty additional experiments conducted at 15 universities in 8 countries including Yale, Columbia, MIT, Harvard, Brown and Stanford show conscious attention and cognitive capacity expand when brainwave activity is attenuated. These experiments conclusively demonstrate that higher states of awareness are accompanied by lower levels of brainwave activity, and that lower states of awareness are accompanied by higher levels of brainwave activity.

Although counterintuitive, it is realized herein that an inverse relationship exists between brainwave activity and cognitive ability. The experiments indicate that reduced brainwave activity is accompanied by higher states of awareness, concentration, focus, mental acuity and cognitive ability. Accordingly, attenuating a subject's brainwave activity will yield a cognitive enhancement.

This section cites 20 neuroscience experiments with human subjects providing evidence that consciousness level varies inversely with brainwave power. The experiments are divided into 2 parts.

Part One presents 3 classes of experiments which demonstrate higher brainwave power accompanies reduced consciousness.
Part Two presents 3 classes of experiments which show that lower brainwave power is correlated with increased consciousness.
Part One—Higher Brainwave Power Accompanies Reduced Consciousness BP↓C↑BP=brainwave power C=consciousness

### 3.1. Anesthesia Experiments

A team of researchers at M.I.T., Harvard, Brown and Boston University led by ShiNung Ching recorded the brainwaves of subjects as they received anesthesia. They found that loss of consciousness was accompanied by an increase in low beta and high alpha band brainwave power. (Ching et al, 2010) (1)

Neuroscientists at Harvard, M.I.T. and Brown led by Patrick Purdon confirmed Ching's results by recording the brainwaves of 10 subjects as they were gradually given anesthesia. They noticed that as the subjects lost consciousness, their brainwave power increased. (Purdon et al, 2013) (2)

### 3.2. Fainting Experiment

A team of scientists in Rome tested 63 patients with a history of fainting, and induced unconsciousness using a tilt table. They observed that loss of consciousness was accompanied by an increase in EEG brainwave amplitude. When patients regained consciousness, their brainwave amplitude diminished. (AMMIRATI et al, 1998) (3)

### 3.3. Exercise Experiments

We can empirically observe that vigorous exercise temporarily reduces a person's cognitive capacity. For example, it much easier to recite the multiplication tables while comfortably seated than while running a hundred-yard dash.

Researchers at Elon University in North Carolina tested 20 subjects during exercise on a recumbent bicycle. They discovered brain EEG activity increased during exercise, and may be related to exercise intensity. Brain EEG activity returned to resting levels quickly after the cessation of exercise. (Bailey et al, 2008) (4)

A team of exercise physiologists in Germany measured 11 subjects during exercises on a treadmill and a stationary bicycle. They found exercising raised alpha and beta brainwave activity. (Schneider et al, 2009) (5)
Part Two—Lower brainwave power accompanies increased consciousness BP↓C↑BP=brainwave power C=consciousness 3.4. Meditation Experiments

If brainwave activity increases when people become less conscious, what does it do when people enter into states of higher awareness? A natural starting point for this inquiry would be to study meditation.

Neuroscience researchers at Yale, Columbia and the University of Oregon tested the brainwaves of 12 subjects during meditation. They deliberately restricted their sample to very experienced meditators from a single practice tradition (mindfulness/insight meditation). This approach was intended to reduce heterogeneity in meditation practices. They found meditation reduced brainwave activity (Brewer et al, 2011). (6)

Subjective experience of meditative states has also been associated with reduced activity in the brain's default mode network in a study of 32 subjects conducted by researchers at the University of Massachusetts and Stanford (van Lutterveld et al, 2017) (7), as well as in four additional experiments cited by van Lutterveld and Brewer in their 2015 paper (8), including Brewer et al, 2011 (6), Pagnoni et al, 2012 (9), Brewer and Garrison 2013 (10) and Garrison et al, 2015 (11). The opposite effect—distracted awareness with higher default mode network activity—has also been observed by Brewer and Garrison, 2013 (10).

3.5. Experiments with Psychoactive Compounds

Searching the neuroscience field for laboratory experiments which measure the brainwaves of people in higher states of consciousness also reveals a large body of literature on experiments with psychoactive compounds, which are known to expand consciousness and promote metacognition.

Neuroscientists from four universities in the UK tested 15 subjects under psilocybin with functional magnetic resonance imaging (fMRI) and magnetoencephalography (MEG). They discovered that expanded states of awareness were accompanied by large decreases in brainwave oscillatory power and reduced neural activity. (Muthukumaraswamy et al, 2013) (12)

Neuroscience researchers at the Imperial College of London and three other UK universities summarized the results of several experiments which used different neuroimaging (brainscan) techniques—functional magnetic resonance imaging (fMRI) and magnetoencephalography (MEG)—to understand how psychedelics change brain functions to alter consciousness. They concluded that consciousness-expanding psychedelics cause brain activity, functional connectivity and oscillatory power to all decrease in brain regions that are normally highly metabolically active. (Carhart-Harris et al, 2014) (13)

Neuroscientists at the Imperial College of London and two other UK universities administered psilocybin to 15 volunteers. They observed profound expansion of consciousness which was accompanied by significantly decreased brain activity. They noticed the magnitude of the reduction in brain activity correlated positively with the intensity of the drug's subjective effects. (Carhart-Harris et al, 2012) (14)

A team of neuroscientists in Switzerland tested the effects of psilocybin on 50 volunteers. They found the mind-expanding drug reduced brainwave current. They also noticed the intensity levels of psilocybin-induced consciousness expansion and insightfulness correlated with desynchronization of brainwaves (which reduces their voltage by wave interference). (Kometer et al, 2015) (15)

Researchers from universities in Spain and Austria tested the effects of the mind-expanding psychoactive beverage ayahuasca on 18 subjects. They found ayahuasca decreased absolute brainwave power across all frequencies. (Riba et al, 2002) (16)

Neuroscientists from four universities in the UK measured the effects of the psychoactive drug MDMA on 25 volunteers. They found MDMA reduced brain activity, and the magnitude of the reductions was highly correlated with the subjective intensity of the drug's mind-expanding effects. (Carhart-Harris et al, 2013) (17)

A study of 58 subjects conducted by Candace Lewis at the University of Zurich found oral psilocybin engendered expanded states of consciousness accompanied by decreased absolute cerebral blood flow in healthy participants. (18)

### 3.6. Intelligence Experiments

Lower brainwave current not only results in higher awareness, it also raises IQ. Scientists at the Ruhr University in Bochum, Germany have discovered that higher IQ individuals have fewer dendrites in their brains. A team of researchers led by Dr. Erhan Genc analyzed the brains of 259 subjects using neurite orientation dispersion and density imaging, which enabled them to measure the amount of dendrites in the cerebral cortex. All participants completed IQ tests which were correlated with their neuroimages. (19)

The results showed that the more intelligent a person is, the fewer dendrite connections there are between the neurons in their cerebral cortex. Using a database from the Human Connectome Project, Dr. Genc's team confirmed these results in a second sample of 500 individuals.

Receptors are located on dendrites. Fewer dendrites means fewer receptors. Fewer receptors yields higher resistance, which makes neurons less excitable. Less excitable neurons fire less often, lowering brainwave activity.

Dr. Genc's report also cites other studies which have shown the brains of highly intelligent people demonstrate less neuronal activity during an IQ test than the brains of average individuals. Neuronal activity is measured in voltage.

One such experiment, conducted by Dr. Richard Haier at University of California Irvine, found significantly lower brain activity in subjects during an abstract reasoning test, as indicated by cortical metabolic rates measured with positron emission tomography (PET). (20)

### 3.7 Summary

It is realized herein that genome editing can be used to increase attention and cognitive resources for five reasons.

1. Human brainwave experiments show conscious awareness and cognitive capacity expand when brainwave activity is reduced.
2. Brainwave activity can be lowered by reducing the accompanying brain currents, since moving electrical currents generate electromagnetic waves (per Ampere's Law).
3. Brain currents can be reduced by raising neuron resistance, since higher resistance impedes the flow of electrical current (per Ohm's Law).
4. Experiments in China and the US have shown that CRISPR can precisely edit genes in neurons.
5. Unconscious brainwave activity can be reduced by lowering neuron excitability. Genome editing can dampen neuron excitability by modifying neurons to raise their electrical resistance, thereby attenuating brainwave activity and yielding expanded cognitive capacity, mental acuity and conscious awareness.

### 4. Receptor Choice

Many kinds of unconscious brainwave activity are vital, but certain types of activity are superfluous. Great care must be exercised in selecting neuron editing strategies which attenuate only unnecessary brainwave activity without interfering with essential neurosignaling pathways.

The present design affects serotonin 2A receptors, which have been extensively studied in 600 drug discovery experiments and are safe to modify in limited dosages. These receptors are most densely expressed in the posterior cingulate cortex; a brain region experimentally correlated with distraction, inattention, mind-wandering and craving. Lowering neural activity in this region will boost attention, focus and mental clarity. The primary gene of interest, HTR2A, is minimally polymorphic and is chemically dissimilar to its neighbors on the chromosome.

Numerous neuroscience experiments associate down-regulating the 5-hydroxytryptamine 2A (5-HT2A) receptor with reduced brainwave power and expanded states of cognitive capacity. Accordingly, the 5-HT2A receptor is a prime candidate for use in genetic cognitive engineering.

### 5. Editing Approach

### 5.1 Feasibility

Chinese researchers are already using CRISPR to reduce neuron activity in mice. A team of scientists at Tsinghua University in Beijing used dCas9-based CRISPR interference (CRISPRi) to efficiently silence genes in neurons, demonstrating that CRISPRi shows superior targeting specificity without detectable off-target activity. (21)

Also, the Max Planck Florida Institute for Neuroscience has demonstrated precise CRISPR editing in mature mouse neurons in vivo regardless of cell maturity, brain region or age. Jun Nishiyama, Takayasu Mikuni, and Ryohei Yasuda used a packaging technique called vSLENDR to provide CRISPR with templates which raise its editing efficacy, achieving extremely efficient results in mouse neurons. They also tested their system in an aged Alzheimer's disease mouse model showing that the vSLENDR technique can be applicable in pathological models even at advanced ages. (22)

Editing RNA as a strategy for treating Alzheimer's has been validated by CRISPR co-inventor Feng Zhang at the Broad Institute at Harvard/MIT. Zhang and his colleagues used the CRISPR Cas13 RNA editor to convert the gene variant APOE4—a risk factor for late-onset Alzheimer's disease—into the non-pathogenic variant APOE2. (23)

### 5.2 Strategy

### 5.2.1 Background

Serotonin (5-Hydroxytryptamine; 5-HT) is a Neurotransmitter that Occupies an Important Place in neurobiology because of its role in many physiologic processes such as cognition, sleep, appetite, thermoregulation, pain perception, hormone secretion, and sexual behavior. Abnormality of the serotonergic system has been implicated in a number of human diseases such as mental depression, obsessive-compulsive disorder, and affective disorders. Like other neurotransmitters, 5-HT is released into the synaptic junction and exerts its effect on specific receptors on the postsynaptic membranes. Based on differential radioligand binding affinities, at least 6 types of 5-HT receptors have been identified: 5-HT-1A, -1B, -1C, -1D, -2, and -3 (summary by Sparkes et al., 1991; see reviews by Peroutka, 1988 and Paoletti et al., 1990).

### 5.2.2 Cloning and Expression

Using a restriction fragment of rat 5-HT2 receptor cDNA, Chen et al. (1992) identified 5-HT2 receptor clones from a human genomic library. The deduced amino acid sequences of the human, mouse, and rat 5-HT2 receptors are highly conserved; all 3 share 90% sequence identity (Chen et al., 1992).

### 5.3.3 Mapping

Sparkes et al. (1991) used a rat cDNA clone for HTR2, which had been shown to cross-hybridize with human and mouse DNA, to map the gene in mouse and man by somatic cell hybrid and in situ hybridization studies. They concluded that the gene is located on chromosome 13q14-q21 in man and on chromosome 14 in the mouse. Hsieh et al. (1990) confirmed the assignment of the HTR2 locus to human chromosome 13 and mouse chromosome 14 by somatic cell hybrid analysis. Furthermore, linkage studies in CEPH families, using a PvuII RFLP detected with the HTR2 probe, showed tight linkage between HTR2 and the locus for esterase D (133280). They concluded that HTR2 is probably between ESD and RB1 (614041). Liu et al. (1991) demonstrated that mouse Htr2 gene is tightly linked to esterase-10 on mouse chromosome 14. A mouse neurologic mutation, agitans (ag), maps to the region of chromosome 14 that, on the basis of syntenic homology, Hsieh et al. (1990) suggested may contain the Htr2 locus.

### 5.3.4 Molecular Genetics

Genomic imprinting describes a parent-of-origin-dependent epigenetic mechanism through which a subset of genes is expressed from only one allele. The allele-specific loss of expression can be polymorphic; that is, it can vary between individuals. Examples of genes that are polymorphically imprinted include the HTR2A gene (Bunzel et al., 1998).

Patients with major depressive disorder (608516) whose treatment is unsuccessful with one medication often have a response when treated with an antidepressant of a different chemical class. McMahon et al. (2006) searched for genetic predictors of treatment outcome in 1,953 patients with major depressive disorder who were treated with the antidepressant citalopram and were prospectively assessed. They detected significant and reproducible association between treatment outcome and a marker in intron 2 of HTR2A, dbSNP rs7997012. Other markers in HTR2A also showed evidence of association with treatment outcome in the total sample. The serotonin-2A receptor, which is encoded by the HTR2A gene, is downregulated by citalopram.

Participants who were homozygous for the A allele had an 18% reduction in absolute risk of having no response to treatment compared with those homozygous for the other allele. The A allele was over 6 times more frequent in white than in black participants, and treatment was less effective among black participants. The A allele may contribute to racial differences in outcomes of antidepressant treatment. Taken together with previous neurobiologic findings, these new genetic data made a compelling case for a key role of HTR2A in the mechanism of antidepressant action.

Lohmueller et al. (2003) performed a metaanalysis of 301 published genetic association studies covering 25 different reported associations. For 8 of the associations, pooled analysis of follow-up studies yielded statistically significant replication of the first report, with modest estimated genetic effects. One of these associations was that of schizophrenia with the C allele of the 102T/C SNP in the HTR2A gene (182135.0001), as first reported by Inayama et al. (1996).

Harvey et al. (2003) investigated whether the agonist serotonin and antagonists loxapine and clozapine have an altered potency for 4 allelic variants (T25N, I197V, A447V, and H452Y) of the human 5HT2A receptor when compared with a wildtype allele. The studies were done by an in vitro functional assay system consisting of an insect cell line that was stably transformed with human wildtype and mutant alleles. This assay system measured release of calcium stores due to receptor activation by agonists and inhibition of this agonist stimulated response by antagonists. They found that the I197V allele required a 2-fold higher concentration of the atypical neuroleptic clozapine to inhibit serotonin stimulation compared to the wildtype receptor (P=0.036). The I197V mutation did not affect the inhibition of serotonin stimulation by the typical neuroleptic loxapine nor did it alter the activation of the receptor by serotonin. The other 3 mutations did not significantly alter the response of the receptor to the agonist serotonin or to the antagonists loxapine and clozapine.

De Quervain et al. (2003) presented evidence that individuals with the H452Y polymorphism performed poorer on memory recall tests than individuals with a normal genotype, suggesting a role for the HTR2A receptor in memory functioning.

Enoch et al. (1998) and Walitza et al. (2002) found an association between the A allele of the -1438G-A promoter polymorphism (182135.0002) and obsessive-compulsive disorder (OCD; 164230).

Holmes et al. (1998) genotyped a total of 211 subjects from a population-based prospective study of psychopathology within late-onset Alzheimer disease (AD; 104300) for the 102T-C polymorphism and the cys23-to-ser polymorphism of the 5-HT-2C receptor gene (312861.0001). Associations were found between the presence of the 102C allele and the presence of both visual and auditory hallucinations. Among 96 AD patients, Assal et al. (2004) found that the 102T allele was associated with agitation/aggression and delusions, but not hallucinations.

The -1438G-A polymorphism has been implicated in other neuropsychiatric disorders such as schizophrenia (181500) (Arranz et al., 1998), seasonal affective disorder (see 608516) (Enoch et al., 1999), alcohol dependence (103780) (Nakamura et al., 1999), and anorexia nervosa (606788) (Collier et al., 1997). Hinney et al. (1997) and Campbell et al. (1998) found no association of the A allele of the -1438G-A polymorphism with anorexia nervosa.

Nakamura et al. (1999) suggested that the A allele of the -1438G-A polymorphism could be associated with restrictive behavior while the G allele could be associated with food and alcohol addiction. Aubert et al. (2000) reported that the -1438G-A polymorphism influences food and alcohol intake in obese (601665) French subjects.

### 5.3.5 Editing Strategy Examples

Strategy 1—Delete the entire HTR2A gene

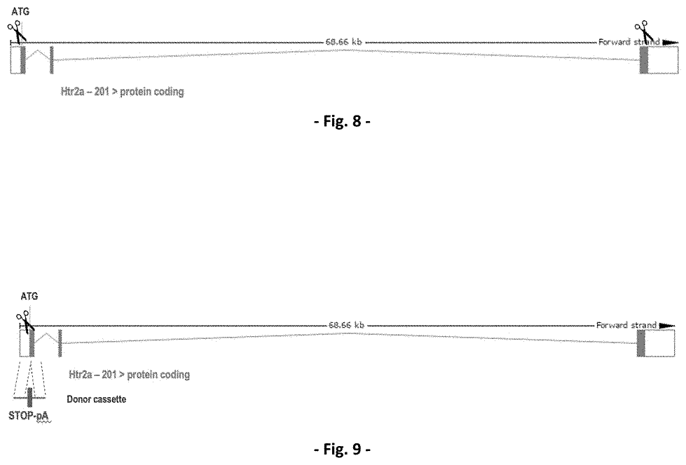

The Htr2a gene encodes a single protein-coding transcript, Htr2a-201. Strategy 1 is designed to produce a deletion of the entire ˜68 kb genomic region. As shown in FIG. 8, CRISPR/Cas9 can be used to make a cut in the first coding exon and a second cut near the termination codon of the Htr2a gene. In the absence of a template, the DSB repair of two breaks may produce an unpredictable number of different alleles, which may complicate genotyping.

Strategy 2—Knock-in of STOP-pA cassette at the start of HTR2A

An insertion of a STOP cassette at the transcription start site (ATG) may be a straightforward strategy. For this purpose, the preferred strategy is to insert a STOP-pA cassette at the start of some fraction, and in some cases, all protein coding transcripts. In some situations, this may be a better alternative to random indel generation or deletion of the entire genomic region. As shown in FIG. 9, CRISPR/Cas9 can be used to make a single cut in the first coding exon and homology-directed repair (HDR) to insert a STOP-pA cassette to terminate transcription of the Htr2a gene. In some instances, such a one-cut approach may be highly efficient and may minimize off-target actions by reducing the number of double-strand DNA breaks. The insertion of a defined homology directed repair template may allow for predictable outcomes and confidence in the design of genotyping assays.

### III. Methodology

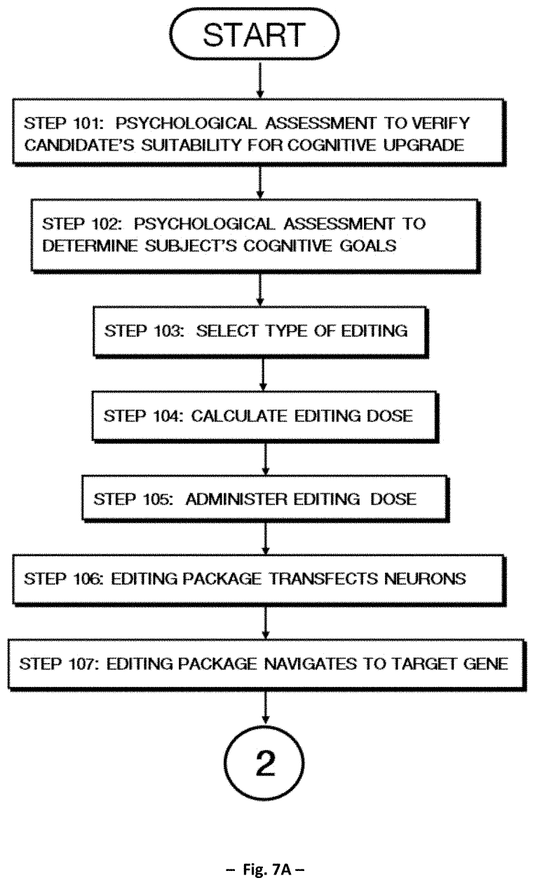

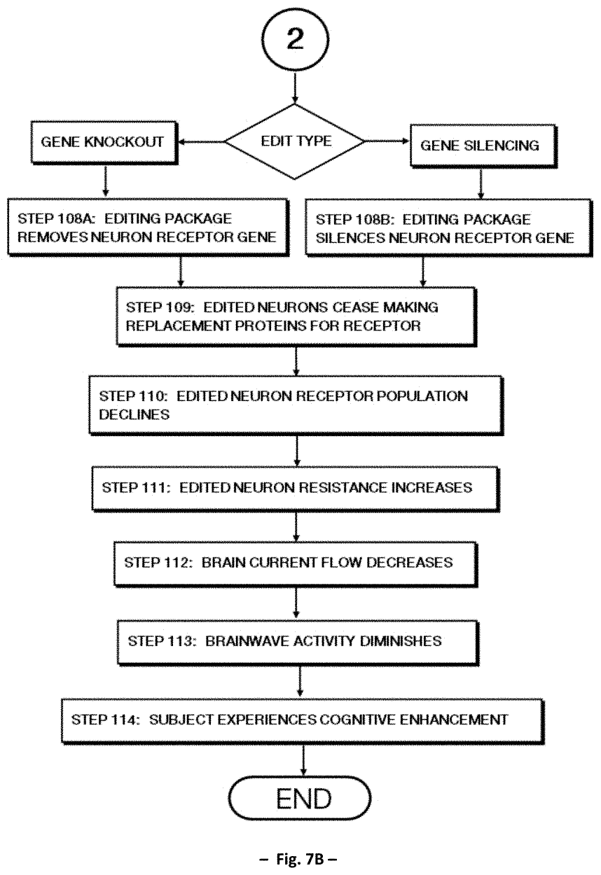

FIGS. 7A and 7B illustrate a method for sustainable human cognitive enhancement. This method employs a gene-editing endonuclease complexed with a neuron-targeting vector and a synthetic guide RNA for lowering the population of 5-hydroxytryptamine 2A receptors in the brain, referred to as an “editing package,” which can be fabricated and manufactured by methods well known in the art. For example, the editing package can comprise a CRISPR-Cas9 endonuclease manufactured by Aldevron and a guide RNA for gene HTR2A manufactured by Synthego carried in adeno-associated viral vectors made by Vector Biosystems. For experiments with mice, a spCas9-type endonuclease may be combined with the guide RNA sequence TGCAATTAGGTGACGACTCGAGG (SEQ ID NO: 1) for targeting the HTR2A gene. Due to the large size of Cas9, each payload may be packaged in a separate vector. A ssODN 200-bp donor sequence repair template may be added to the guide RNA vector to ensure that stop codons are inserted into exon 1 of the HTR2A gene.

### Step 101: Psychological Assessment to Verify Candidate's Suitability for Cognitive Upgrade

It is realized herein that general-purpose genetic cognitive enhancement is suitable for adults in sound mental and emotional health. The process begins with a psychological assessment to screen out candidates who do not meet this criterion, for example, individuals with alcohol or substance abuse, bipolar disorder, depression, schizophrenia or other psychological conditions or disorders.

The assessment also ensures the candidate is not currently taking any drugs, medications or substances that could interfere with the normal, natural functioning of their brain; for example, alcohol, caffeine, nicotine, cannabis, nootropics, ginseng or other similar substances or herbal preparations.

Candidates who satisfactorily meet the psychological assessment criteria are accepted as subjects for cognitive enhancement.

Step 102: Psychological Assessment to Determine Subject's Cognitive Goals

The second step is a psychological assessment to ascertain the subject's cognitive enhancement goals. This assessment covers topics such as whether the cognitive upgrade is to be permanent or temporary, whether it will be reversible and, if temporary, the number of years the upgrade shall have effect.

### Step 103: Select Type of Editing

The type of editing is chosen based on the outcome of the assessment. Gene silencing may be used when the subject wishes to have a temporary or reversible cognitive upgrade, and gene knockout may be selected when the subject wishes to have a permanent upgrade.

### Step 104: Calculate Editing Dose

### 1. Background

As shown in Table 1, chemical and genetic doses work much differently. A chemical dose's effects occur at the individual receptor level, whereas a genetic dose's effects occur at the neuron level. Hence, a chemical dose can affect all, some or none of a neuron's 5-HT2A receptors, whereas a genetic dose will affect all or substantially all of a neuron's 5-HT2A receptors.

TABLE 1

| ATTRIBUTE | CHEMICAL DOSE | GENETIC DOSE |
|---|---|---|
| Dose target: | Neuron receptor | Neuron gene |
| Dose disables: | One 5-HT2A | One gene in one neuron |
| | receptor | having many 5-HT2A |
| | | receptors |
| Amount of dose | A miniscule percent | Almost 100%. Genetic edits |
| that reaches | (e.g., 0.01%) | can be precisely targeted to |
| brain: | | brain neurons using |
| | | navigational guides or vectors. |
| Dose is absorbed | 5-HT2A receptors | Neurons having between 0 |
| by: | on neurons. | and 1000+ 5-HT2A receptors |
| Editing | N/A | Current genetic editing |
| efficiency | | efficiency for individual |
| | | genes is approximately 100%. |

### 2. Formula

A variety of formulas can be developed to calculate dosages based on different subject needs and applications. Given below is a simplified example of a formula for calculating a genetic cognitive enhancement dose which is equivalent to a given chemical cognitive enhancement dose which temporarily disables 5-HT2A receptors. Open source neuron simulation models, such as Yale's NEURON model, can be used to calculate precise dosages.

1. Receptors affected per Chemical Dose (RCD)

Calculate the number of 5-HT2A receptors affected by a known chemical dose (CDR).

a) Known chemical dose=n molecules
b) Approximately y % of dose reaches the brain
c) n molecules x y %=m molecules
d) 1 molecule affects 1 receptor
e) The number of 5-HT2A receptors affected by a chemical dose (RCD)=m receptors.

### 2. Equivalent Number of Neurons (ENN)

Calculate the number of neurons whose total combined 5-HT2A receptor population equals the number of receptors affected by a chemical dose (RCD).

a) Receptors per dendrite=Rd
b) Dendrites per neuron=Dn
c) Receptors per neuron=Rn. Rn=Rd×Dn
d) Average percent of receptors which are 5-HT2A receptors=p %
e) Average number of 5-HT2A receptors per neuron=Rh. Rh=Rn×p %
f) From step 1, receptors affected by chemical dose (RCD)=m receptors.
g) m receptors divided by Rh 5-HT2A receptors per neuron=s neurons
h) The number of neurons whose combined total 5-HT2A receptor population equals the number of receptors affected by a known chemical dose is s neurons. This is the Equivalent Number of Neurons (ENN).

### 3. Editing Efficiency Factor (EEF)

Include the effects of factors which constrain genetic editing efficiency.

a) Genetic Editing Efficiency (GEE %) is e % with current technology, meaning that e % of the edits which are absorbed by neurons will be effective. This percent is lower with catalytically-inactive endonuclease gene editors which have a one-time use than it is with catalytically-active editors which can edit target genes in both chromosome pairs.
b) Neurons transfected With Receptor (NWR %): Although the 5-HT2A receptor is widely expressed in the neural cortex, some of the neurons which absorb the genetic dose will not have the receptor. The Neurons With Receptor (NWR %) factor is g %, meaning that g % of neurons which absorb the genetic dose possess 5-HT2A receptors.

### c) Editing Efficiency Factor (EFF)=GEE %×NWR %.

### 4. Genetic Dose

The genetic dose which is equivalent to the chemical dose is calculated as follows:

$$\frac{Equivalent Number of Neurons ( ENN )}{Editing Efficiency Factor ( EFF )}$$

### Step 105: Administer Editing Dose

Editing package doses can be administered to subjects via oral, sublingual, intranasal or transdermal application or through other methods well known in the art.

### Step 106: Editing Package Transfects Neurons

The vector in the editing package transfects CNS neurons.

### Step 107: Editing Package Navigates to Target Gene

Once inside the cell, the guide RNA in the editing package navigates the package to gene HTR2A and attaches itself to the gene's location on chromosome 13. This can be accomplished with considerable precision using currently available gene editing guides such as single-guide RNA (sgRNA).

### Referring to FIG. 7B:

### Step 108A: Editing Package Removes Neuron Receptor Gene

If the subject has chosen gene knockout, then once attached to chromosome 13, the gene-editing endonuclease in the editing package deletes gene HTR2A from the chromosome.

### Step 108B: Editing Package Silences Neuron Receptor Gene

If the subject has chosen gene silencing, then once attached to chromosome 13, the gene-silencing endonuclease in the editing package silences gene HTR2A by blocking its RNA transcription.

### Step 109: Edited Neurons Cease Making Replacement Proteins for Receptor

Gene HTR2A supplies neurons with the blueprints for manufacturing cellular proteins which are used to build 5-hydroxytryptamine 2A receptors. When this gene is deleted from the chromosome or its RNA transcription is blocked, the neuron stops making the proteins needed to replace its 5-hydroxytryptamine 2A receptors.

### Step 110: Edited Neuron Receptor Population Declines

There are fifty different types of neuron receptors, and neurons typically contain a mixture of multiple types of receptors. When a neuron's 5-hydroxytryptamine 2A receptors are not replaced, its overall number of receptors declines.

### Step 111: Edited Neuron Resistance Increases

A neuron's receptor sites serve as doorways which receive the flow of electrically-charged ions into the neuron. A neuron will fill its cellular reservoir with incoming charged ions more quickly if it has a larger number of receptor sites.

TABLE 2

| Receptor | | | Current |
|---|---|---|---|
| Sites | Resistance | Excitability | Flow |
| [symbol] | [symbol] | [symbol] | [symbol] |
| [symbol] | [symbol] | [symbol] | [symbol] |

Referring to Table 2, increasing the number of a neuron's receptor sites adds more channels for incoming ions to flow into, similar to adding more lanes to a freeway. This gives the neuron lower electrical resistance, which makes it more easily excitable.

Conversely, decreasing a neuron's receptor population reduces the number of pipes for incoming ions to flow into, like closing lanes on a freeway. This raises the neuron's electrical resistance, making it harder to excite.

A neuron's resistance can be modified by changing its number of receptor sites. Reducing a neuron's number of receptor sites by removing its 5-HT2A receptors decreases the number of doorways or pipes for electrically-charged ions to flow through, thereby increasing the neuron's resistance. This decelerates the flow of electrons from one neuron to another.

### Step 112: Brain Current Flow Decreases

Raising a neuron's resistance lowers its conductivity. Less-conductive neurons have a lower capacity for carrying the flow of electrical current in the brain.

### Step 113: Brainwave Activity Diminishes

A moving electrical current generates an electromagnetic wave (per Ampere's Law). Flowing electrons in the brain generate brainwaves. When the flowing electrons slow down, so does brainwave activity.

Less-conductive, less-excitable neurons require more time to fill their cellular reservoirs with enough electrically-charged ions to cause them to fire. Hence, they fire less frequently. Lower neuron activity reduces brainwave activity.

### Step 114: Subject Experiences Cognitive Enhancement

Numerous scientific studies have conclusively demonstrated reduced brainwave activity is correlated with higher states of awareness, concentration, focus, mental acuity and cognitive ability. Accordingly, attenuating the subject's brainwave activity will yield a cognitive enhancement.

Although specific embodiments of the invention have been disclosed herein in detail, it is to be understood that this is for the purpose of illustrating the invention, and should not be construed as necessarily limiting the scope of the invention, since it is apparent that many changes can be made to the disclosed methods by those skilled in the art to suit particular applications.

### REFERENCES

(1) Thalamocortical model for a propofol-induced alpha-rhythm associated with loss of consciousness
ShiNung Ching, et al
Proceedings of the National Academy of Sciences vol. 107 no. 52, 22665-22670
http://www.pnas.org/content/107/52/22665.abstract
(2) Electroencephalogram signatures of loss and recovery of consciousness from propofol
Patrick Purdon, et al
Proceedings of the National Academy of Sciences vol. 110 no. 12, E1142-E1151
http://www.pnas org/content/110/12/E1142.full
(3) Electroencephalographic Correlates of Vasovagal Syncope Induced by Head-Up Tilt Testing
Fabrizio Ammirati, et al
Stroke 1998 November; 29(11):2347-51
http://www.ncbi.nlm.nih.gov/pubmed/9804646
(4) Changes in EEG During Graded Exercise on a Recumbent Cycle Ergometer
Stephen P. Bailey, et al
Journal of Sports Science and Medicine (2008) 7, 505-511
http://www.ncbi.nlm.nih.gov/pmc/articles/PMC3761919/
(5) Changes in brain cortical activity measured by EEG are related to individual exercise preferences.
S. Schneider, et al
Journal of Physiological Behavior 2009 Oct. 19; 98(4):447-52.
http://www.ncbi.nlm.nih.gov/pubmed/19643120
(6) Meditation experience is associated with differences in default mode network activity and connectivity
Judson A. Brewer, et al
Proceedings of the National Academy of Sciences USA 2011 Dec. 13; 108(50): 20254-20259.
http://www.ncbinlmnih.gov/pmc/articles/PMC3250176/
(7) Source-space EEG neurofeedback links subjective experience with brain activity during effortless awareness meditation
Remko van Lutterveld et al, 2017
Neuroimage, 2017 May 1; 151:117-127. doi: 10.1016/
https://www.ncbi.nlm.nih.gov/pubmed/26921712
(8) Neurofeedback from the Posterior Cingulate Cortex as a Mental Mirror for Meditation.
Remko van Lutterveld and Judson Brewer
Biofeedback: Fall 2015, Vol. 43, No. 3, pp. 117-120.
http://dx.doi.org/10.5298/1081-5937-43.3.05
(9) Dynamical Properties of BOLD Activity from the Ventral Posteromedial Cortex Associated with Meditation and Attentional Skills
Giuseppe Pagnoni, 2012
Journal of Neuroscience 11 Apr. 2012, 32 (15) 5242-5249;
http://www.jneurosci.org/content/32/15/5242.full
(10) Effortless awareness: using real time neurofeedback to investigate correlates of posterior cingulate cortex activity in meditators' self-report
Kathleen A. Garrison, et al 2013
Frontiers in Human Neuroscience v.7; 2013 Aug. 6 PMCID: PMC3734786
https://www.ncbi.nlm.nih.gov/pmc/articles/PMC3734786/
(11) Meditation leads to reduced default mode network activity beyond an active task
Kathleen A. Garrison, et al 2015
Cognitive, Affective, & Behavioral Neuroscience
September 2015, Volume 15, Issue 3, pp 712-720
https://www.ncbi.nlm.nih.gov/pmc/articles/PMC4529365/
(12) Broadband Cortical Desynchronization Underlies the Human Psychedelic State
Suresh D. Muthukumaraswamy, et al
Journal of Neuroscience, 18 Sep. 2013, 33(38): 15171-15183;
http://www.jneurosci.org/content/33/38/15171.full
(13) The entropic brain: a theory of conscious states informed by neuroimaging research with psychedelic drugs
Robin L. Carhart-Harris, et al
Frontiers in Human Neuroscience 2014; 8: 20.
http://www.ncbi.nlm.nih.gov/pmc/articles/PMC3909994/
(14) Neural correlates of the psychedelic state as determined by fMRI studies with psilocybin
Robin L. Carhart-Harris, et al
Proceedings of the National Academy of Sciences USA 2012 Feb. 7; 109(6): 2138-2143
http://www.ncbinlmnih.gov/pmc/articles/PMC3277566/
(15) Psilocybin-induced spiritual experiences and insightfulness are associated with synchronization of neuronal oscillations
Michael Kometer, et al
Psychopharmacology (Berlin). 2015 October; 232(19):3663-76.
http://www.ncbi.nlm.nih.gov/pubmed/26231498
(16) Topographic pharmaco-EEG mapping of the effects of the South American psychoactive beverage ayahuasca in healthy volunteers
Jordi Riba, et al
British Journal of Clinical Pharmacology 2002 June; 53(6): 613-628.
http://www.ncbi.nlm.nih.gov/pmc/articles/PMC1874340/
(17) The effects of acutely administered 3,4-methylenedioxymethamphetamine on spontaneous brain function in healthy volunteers
Robin Carhart-Harris, et al
International Journal of Neuropsychopharmacology 2013 10.1017/S1461145713001405
Biological Psychiatry 2015 Oct. 15; 78(8):554-62
http://www.ncbi.nlm.nih.gov/pubmed/24495461
(18) Two dose investigation of the 5-HT-agonist psilocybin on relative and global cerebral blood flow
Candace R. Lewis, Katrin H. Preller, et al
NeuroImage Volume 159, 1 Oct. 2017, Pages 70-78
https://doi.org/10.1016/j.neuroimage.2017.07.020
(19) Diffusion markers of dendritic density and arborization in gray matter predict differences in intelligence
Erhan Genc, Christoph Fraenz, et al
Nature Communications, 2018, volume 9, Article number 1905
doi:10.1038/s41467-018-04268-8
http://www.nature.com/articles/s41467-018-04268l-8
https://www.ncbi.nlm.nih.gov/pmc/articles/PMC5954098/
(20) Cortical glucose metabolic-rate correlates of abstract reasoning and attention studied with positron emission tomography. Haier R J, et al, Intelligence. 1988; 12:199-217.
doi: 10.1016/0160-2896(88)90016-5
https://doi.org/10.1016/0160-2896(88)90016-5
(21) CRISPR interference-based specific and efficient gene inactivation in the brain
Yi Zheng, Wei Shen, Jian Zhang, Bo Yang, Yao-Nan Liu, Huihui Qi, Xia Yu, Si-Yao Lu, Yun Chen
Nature Neuroscience volume 21, pages 447-454 (2018)
https://www.nature.com/articles/s41593-018-0077-5
(22) Virus-Mediated Genome Editing via Homology-Directed Repair in Mitotic and Postmitotic Cells in Mammalian Brain.
Jun Nishiyama*, Takayasu Mikuni*, and Ryohei Yasuda
Neuron, Oct. 19 2017,
DOI: 10.1016/j.neuron.2017.10.004
https://www.maxplanckflorida.org/news-and-media/news/gene-editing-in-the-brain-gets-a-major-upgrade/
(23) CRISPR pioneer Zhang targets brain diseases with new RNA-editing system
Fierce Biotech, Arlene Weintraub, Jul. 11, 2019
https://www.fiercebiotech.com/research/crispr-pioneer-zhang-targets-brain-diseases-new-rna-editing-system

What is claimed is:

1. A method for general-purpose cognitive enhancement of a subject comprising administering a gene-editing endonuclease complexed with a neuron-targeting vector and a synthetic guide RNA to the subject to lower the population of 5-hydroxytryptamine 2A receptors in the brain.

2. The method of claim 1, wherein the gene-editing endonuclease is a catalytically active endonuclease which lowers the population of 5-hydroxytryptamine 2A receptors in the brain of the subject by deleting all or part of the HTR2A gene from CNS neurons of the brain of the subject.

3. The method of claim 1, wherein the gene-editing endonuclease is a catalytically inactive endonuclease which lowers the population of 5-hydroxytryptamine 2A receptors in the brain of the subject by interfering with the transcription or translation of the HTR2A gene into cellular proteins for building 5-hydroxytryptamine 2A receptors.

4. A method of cognitive enhancement of a subject comprising the steps of:
conducting a psychological assessment of the subject to determine suitability for cognitive enhancement;
selecting a subject;
selecting an appropriate gene-editing endonuclease;
administering a suitable dose of the gene-editing endonuclease with a neuron-targeting vector and a synthetic guide RNA to the subject.

5. The method of claim 4, wherein the step of conducting a psychological assessment of the subject to determine suitability for cognitive enhancement includes assessing a subject for any one or more of alcohol or substance abuse, bipolar disorder, depression, schizophrenia and/or other psychological conditions or disorders.

6. The method of claim 5, wherein the step of selecting a subject includes selecting a subject who does not suffer from one or more of alcohol or substance abuse, bipolar disorder, depression, schizophrenia and/or other psychological conditions or disorders.

7. The method of claim 4, wherein the step of conducting a psychological assessment of the subject to determine suitability for cognitive enhancement includes assessing a subject for use of drugs, medications or substances that could interfere with the functioning of the brain of the subject.

8. The method of claim 7, wherein the step of selecting a subject includes selecting a subject who is not using any one or more of alcohol, caffeine, nicotine, cannabis, nootropics, ginseng and/or other similar substances or herbal preparations.

9. The method of claim 4, wherein the step of selecting an appropriate gene-editing endonuclease includes selecting a catalytically active gene-editing endonuclease.

10. The method of claim 4, wherein the step of selecting an appropriate gene-editing endonuclease includes selecting a catalytically inactive gene-editing endonuclease.

11. A method of cognitive enhancement of a human subject comprising the steps of:
conducting a psychological assessment of the subject to determine suitability for cognitive enhancement;
selecting a subject;
selecting either temporary or permanent cognitive enhancement;
preparing a gene-editing package targeting the HTR2A gene for administration to the subject;
administering a suitable dose of the gene-editing package to the subject.

12. The method of claim 11, wherein the step of conducting a psychological assessment of the subject to determine suitability for cognitive enhancement includes assessing a subject for any one or more of alcohol or substance abuse, bipolar disorder, depression, schizophrenia and/or other psychological conditions or disorders.

13. The method of claim 12, wherein the step of selecting a subject includes selecting a subject who does not suffer from one or more of alcohol or substance abuse, bipolar disorder, depression, schizophrenia and/or other psychological conditions or disorders.

14. The method of claim 11, wherein the step of conducting a psychological assessment of the subject to determine suitability for cognitive enhancement includes assessing a subject for use of drugs, medications or substances that could interfere with the functioning of the brain of the subject.

15. The method of claim 14, wherein the step of selecting a subject includes selecting a subject who is not using any one or more of alcohol, caffeine, nicotine, cannabis, nootropics, ginseng and/or other similar substances or herbal preparations.

16. The method of claim 11, wherein the gene-editing package for administration to the subject includes a gene-editing endonuclease complexed with a neuron-targeting vector and a synthetic guide RNA targeted to the HTR2A gene.

17. The method of claim 16, wherein the gene-editing endonuclease is a catalytically active gene-editing endonuclease.

18. The method of claim 16, wherein the gene-editing endonuclease is a catalytically inactive gene-editing endonuclease.

19. The method of claim 16, wherein the neuron-targeting vector is an adeno-associated viral vector.

20. The method of claim 16, wherein the synthetic guide RNA further comprises a donor sequence repair template to add a stop codon to exon 1 of the HTR2A gene.


SEQUENCE LISTING (1 sequence)

SEQ ID NO: 1 - DNA, 23 bp, Artificial Sequence
  DNA template for transcription to guide RNA sequence
tgcaattaggtgacgactcgagg